SEQUENCE LISTING (3 sequences)

SEQ ID NO: 1 - protein, 9 aa, Artificial Sequence
  Chemically synthesized
LAGLIDADG

SEQ ID NO: 2 - protein, 13 aa, Artificial Sequence
  Chemically synthesized
GSKTKEGVVHVTT

SEQ ID NO: 3 - DNA, 41 bp, Artificial Sequence
  Chemically synthesized
gctccaaaaccaaggagggagtggtgcatggtgtgacaaca

(19) United States
(12) Patent Application Publication
(10) Pub. No.: US 2020/0165635 A1
(43) Pub. Date: May 28, 2020

(54) MEANS AND METHODS FOR SELECTING TRANSFORMED CELLS

(71) Applicant: UNIVERSITE DU LUXEMBOURG, Luxembourg (LU)

(72) Inventors: Jonathan Arias FUENZALIDA, Belvaux (LU); Javier JARAZO, Belvaux (LU); Jens SCHWAMBORN, Belvaux (LU); Xiaobing QING, Belvaux (LU)

(21) Appl. No.: 16/073,005

(22) PCT Filed: Jan. 30, 2017
(86) PCT No.: PCT/EP2017/051889

§ 371 Date: Jul. 26, 2018

(30) Foreign Application Priority Data
Jan. 28, 2016 (LU) 92964

(51) Int. Cl. C12N 15/90; C12N 15/79

(52) U.S. Cl. CPC C12N 15/902; C12N 2800/10; C12N 15/79

(57) ABSTRACT

The present invention relates to a nucleic acid molecule at least one nucleotide sequence encoding a selection marker indicating homologous recombination in a eukaryotic cell and at least one nucleotide sequence encoding a selection marker indicating heterologous recombination in said eukaryotic cell. The present invention also relates to a composition of matter comprising at least two nucleic acid molecules of the invention. The present invention further relates to in vitro methods for enriching or producing eukaryotic cells which are modified by homologous recombination.

Targeted gene inactivation via homologous recombination is a powerful method capable of providing conclusive information for evaluating gene function. However, the use of this technique has been hampered by several factors, including the low efficiency at which engineered constructs are correctly inserted into the chromosomal target site, the need for time-consuming and labor-insensitive selection/screening strategies, and the potential for adverse mutagenic effects.

However, meganucleases, Zinc-finger nucleases (ZFNs) and transcription activator-like effector nucleases (TALENs) comprise a powerful class of tools that are redefining the boundaries of biological research. These chimeric nucleases are composed of programmable, sequence-specific DNA-binding modules linked to a non-specific DNA cleavage domain. ZFNs and TALENs enable a broad range of genetic modifications by inducing DNA double-strand breaks that stimulate error-prone non-homologous end joining (NHEJ) or homology-directed repair (HDR) at specific genomic locations. The emergence of clustered regulatory interspaced short palindromic repeat (CRISPR)/Cas-based RNA-guided DNA endonucleases has even broadened the tool box for genome editing.

Meganucleases, ZFNs and TALENs are based on the use of engineered nucleases composed of sequence-specific DNA-binding domains fused to a non-specific DNA cleavage module. These chimeric nucleases enable efficient and precise genetic modifications by inducing targeted DNA double-strand breaks (DSBs) that stimulate the cellular DNA repair mechanisms, including NHEJ and HDR. The versatility of this approach is facilitated by the programmability of the DNA-binding domains that are derived from zinc-finger and transcription activator-like effector (TALE) proteins. This combination of simplicity and flexibility has catapulted ZFNs and TALENs to the forefront of genetic engineering. Nowadays, because of its fast and convenient applicability CRISPR/Cas has become the front-running technology for genome editing.

However, though the genome editing technology is widely and efficiently applicable, the screening and selection of desired clones is nevertheless labor- and time-sensitive requiring, e.g. PCR techniques, sequencing or the like in order to verify desired clones. In fact, the current protocols to derive genome edited lines require the screening of a great number of clones to obtain one lacking random integration or on locus NHEJ containing alleles (Ran et al., Nat. Protoc. 8, 2281-2308 (2013), Sander and Joung, Nat. Biotechnol. 32, 347-355 (2014)).

There is thus still a need for means and methods allowing the selection of transformed cells having the desired genotype. The present application addresses this need and thus provides means and methods for selecting transformed cells having the desired genotype.

Specifically, the present application provides means and methods for streamlining the gene editing process by incorporating reporters to reduce hands on time by automating/simplifying the screening process. Accordingly, the present application makes use of a negative and positive selection module. While the negative selection module allows screening and sorting out undesired random/off-site modified transformed cells, the positive selection module allows the identification of desired on-site modified cells.

By way of example, fluorescent proteins are used as selection markers on the nucleic acid molecule used in gene editing as a donor DNA molecule, wherein different fluorescent proteins are used for positive and negative selection such that both fluorescent proteins are optically discriminable, e.g. in Fluorescent-activated cell sorting (FACS), flow cytometry or fluorescence microscopy. The marker used for positive selection is flanked 5′ and 3′ by nucleotide sequences that are homologous to nucleotide sequences of a nucleic acid sequence of interest comprised by eukaryotic cells, such as mammalian cells or plant cells (homology arms) and thus is indicative of homologous recombination as it is integrated together with the flanking homology arms. A cell comprising the positive selection marker is therefore likely to be a desired on-site (or in locus) modified cells. The marker used for negative selection, not comprised in the region flanked 5′ and 3′ by homology arms, is likely to be integrated in the cellular genome upon heterologous recombination, only, and thus indicates an unwanted genetic modification, allowing to detect off-site (or out-of locus) modified cells.

A preferred embodiment of the technique described herein is sometimes called Fluorescence Assisted Genome Editing (FACE). FACE allows to derive correctly edited clones carrying a positive selection fluorescent marker and to exclude non-edited, random integrations and on-target allele NHEJ-containing cells from the correctly edited polyclonal population. Specifically, the combined use of two nucleic acid molecules each comprising different nucleotide sequences encoding different fluorescent proteins in the positive selection modules loaded onto specific homology arms (mutant/mutant, wild-type/wild-type or mutant/wild-type) allows to deterministically predict the outcome of the modification as designed, thereby giving rise to bi-allelically targeted homozygotes and heterozygote cell populations.

Accordingly, The means and methods of the present application allow the cell population, polyclones or clones that have undergone removal of the positive selection module to be enriched by the selection of cells that lost the optical, e.g. fluorescence signal by, e.g. FACS. After the removal of the positive selection module, e.g. homozygous gene corrected edited lines, homozygous mutant genome edited lines, heterozygous mutant genome edited lines or heterozygous gene corrected edited lines can be subcloned or used for phenotypic characterization, drug screening or cell therapy.

It must be noted that as used herein, the singular forms “a”, “an”, and “the”, include plural references unless the context clearly indicates otherwise. Thus, for example, reference to “an expression cassette” includes one or more of the expression cassettes disclosed herein and reference to “the method” includes reference to equivalent steps and methods known to those of ordinary skill in the art that could be modified or substituted for the methods described herein.

Unless otherwise indicated, the term “at least” preceding a series of elements is to be understood to refer to every element in the series. Those skilled in the art will recognize, or be able to ascertain using no more than routine experimentation, many equivalents to the specific embodiments of the invention described herein. Such equivalents are intended to be encompassed by the present invention.

Throughout this specification and the claims which follow, unless the context requires otherwise, the word “comprise”, and variations such as “comprises” and “comprising”, will be understood to imply the inclusion of a stated integer or step or group of integers or steps but not the exclusion of any other integer or step or group of integer or step. When used herein the term “comprising” can be substituted with the term “containing” or sometimes when used herein with the term “having”.

When used herein “consisting of” excludes any element, step, or ingredient not specified in the claim element. When used herein, “consisting essentially of” does not exclude materials or steps that do not materially affect the basic and novel characteristics of the claim. In each instance herein any of the terms “comprising”, “consisting essentially of” and “consisting of” may be replaced with either of the other two terms.

The term “about” or “approximately” as used herein means within 20%, preferably within 10%, and more preferably within 5% of a given value or range. It includes also the concrete number, e.g., about 20 includes 20.

Unless otherwise defined herein, scientific and technical terms used in connection with the present invention shall have the meanings that are commonly understood by those of ordinary skill in the art. Further, unless otherwise required by context, singular terms shall include pluralities and plural terms shall include the singular. The methods and techniques of the present invention are generally performed according to conventional methods well-known in the art. Generally, nomenclatures used in connection with techniques of biochemistry, enzymology, molecular and cellular biology, microbiology, genetics and protein and nucleic acid chemistry and hybridization described herein are those well-known and commonly used in the art.

The methods and techniques of the present invention are generally performed according to conventional methods well-known in the art and as described in various general and more specific references that are cited and discussed throughout the present specification unless otherwise indicated. See, e. g., Sambrook et al., Molecular Cloning: A Laboratory Manual, 3rd ed., Cold Spring Harbor Laboratory Press, Cold Spring Harbor, N. Y. (2001); Ausubel et al., Current Protocols in Molecular Biology, J, Greene Publishing Associates (1992, and Supplements to 2002); Handbook of Biochemistry: Section A Proteins, Vol 11976 CRC Press; Handbook of Biochemistry: Section A Proteins, Vol II 1976 CRC Press. The nomenclatures used in connection with, and the laboratory procedures and techniques of, molecular and cellular biology, protein biochemistry, enzymology and medicinal and pharmaceutical chemistry described herein are those well-known and commonly used in the art.

The present invention provides a nucleic acid molecule comprising at least one nucleotide sequence encoding a selection marker indicating homologous recombination when integrated in the sequence of interest comprised in a eukaryotic cell, such as a mammalian or plant cell, and at least one nucleotide sequence encoding a selection marker indicating heterologous recombination when not integrated in the sequence of interest comprised in said eukaryotic cell, wherein the selection markers when being expressed are optically discriminable, e.g. in FACS or any fluorescence guided capture, and wherein the nucleotide sequence encoding a selection marker indicating homologous recombination in a eukaryotic cell is flanked 5′ and 3′ by nucleotide sequences that are homologous to nucleotide sequences of a nucleic acid sequence of interest comprised by eukaryotic cell.

A eukaryotic cell when used herein may preferably be a mammalian cell or plant cell. A mammalian cell may preferably be a cell from a human, dog, cat, cow, swine, horse, sheep, goat, rabbit, mouse or rat, with human being preferred. A preferred human cell is a stem cell or induced pluripotent stem cell. The human cells may be obtained from a healthy human or a human suffering from a disease, such as Parkinson disease (PD) or Alzheimer disease (AD). The human cell and any other mammalian cell may be from a cell line, e.g. a deposited cell line or a commonly available cell line.

The term “nucleic acid molecule” or “nucleotide sequence” as used herein refers to a polymeric form of nucleotides (i.e. polynucleotide) which are usually linked from one deoxyribose or ribose to another. The term “nucleic acid molecule” preferably includes single and double stranded forms of DNA or RNA. A nucleic acid molecule may include both sense and antisense strands of RNA (containing ribonucleotides), cDNA, genomic DNA, and synthetic forms and mixed polymers of the above. They may be modified chemically or biochemically or may contain non-natural or derivatized nucleotide bases, as will be readily appreciated by those of skill in the art. Such modifications include, for example, labels, methylation, substitution of one or more of the naturally occurring nucleotides with an analog, internucleotide modifications such as uncharged linkages (e.g., methyl phosphonates, phosphotriesters, phosphoramidates, carbamates, etc.), charged linkages (e.g., phosphorothioates, phosphorodithioates, etc.), pendent moieties (e.g., polypeptides), intercalators (e.g., acridine, psoralen, etc.), chelators, alkylators, and modified linkages (e.g., alpha anomeric nucleic acids, etc.) Also included are synthetic molecules that mimic polynucleotides in their ability to bind to a designated sequence via hydrogen bonding and other chemical interactions. Such molecules are known in the art and include, for example, those in which peptide linkages substitute for phosphate linkages in the backbone of the molecule. In a preferred embodiment the term “nucleic acid molecule” to be introduced into a cell refers to DNA and even more preferred to double stranded DNA, whereas a nucleic acid being an expression product is preferably a RNA.

The term “selection marker”, “selectable marker” or “marker” are used interchangeably herein and refer to a gene introduced into a cell that confers a trait suitable for selection of cells when being expressed, for e.g. successful transfection or a specific genetic modification of a cell. A selection marker preferably provides the cell with a phenotype that is optically detectable via FACS, flow cytometry or fluorescence microscopy, such as e.g. fluorescent proteins.

The term “indicating” and its grammatical variants, such as “indicative for” when used in the context of a selection marker indicating homologous or heterologous recombination, respectively, does not mean that the selection marker is indicative that a homologous or heterologous recombination indeed occurred. Rather, said term means that the selection marker provides a skilled artisan with a reasonable likelihood/probability that homologous or heterologous recombination, respectively, occurred. Put differently, the selection marker is an indicator for a homologous or heterologous recombination, respectively, but is not a proof for homologous or heterologous recombination, respectively.

A selection marker indicating homologous recombination when integrated in the sequence of interest comprised in a eukaryotic cell is thus indicative that homologous recombination occurred, while a selection marker indicating heterologous recombination when not integrated in the sequence of interest comprised in a eukaryotic cell is indicative to detect and exclude recombination events outside of the sequence of interest comprised by a eukaryotic cell.

The term “homologous recombination” as used herein refers to homology-directed repair (HDR) which is a template-dependent pathway for DNA double-strand break repair. By supplying a homology-containing donor template, preferably along with a site-specific nuclease, HDR faithfully inserts the donor molecule at the targeted locus. This approach enables the insertion of single or multiple transgenes, as well as single nucleotide substitutions. Thus, the present invention preferably employs homologous recombination for site-specific genetic modification of the nucleic acid sequence of interest comprised by the eukaryotic cell by integration of the nucleic acid molecule of the invention. To this end, the nucleic acid molecule of the invention comprises nucleotide sequences that are homologous to nucleotide sequences of a nucleic acid sequence of interest comprised by the eukaryotic cell. The homologous nucleotide sequences comprised by the nucleic acid molecule of the invention flank the selection marker which indicates homologous recombination 5′ and 3′ and are thus called “homology arms”. The homology arms direct the nucleic acid molecule of the invention to the desired nucleotide sequence of interest comprised by the eukaryotic cell and thus mediate the site specific integration. As the homology arms are integrated upon homologous recombination, the sequence flanked by the homology arms (i.e. the sequence situated between the homology arms), e.g. the selection marker indicating homologous recombination or any other nucleotide sequence, will also be integrated. The homology arms do preferably not comprise any repetitive element. Without being bound by theory, excluding repetitive elements from the homology arms increases homology directed repair efficiency and decreases the rate of random integration. However, the homology arms can also comprise one or more mismatches compared to the nucleic acid sequence of interest comprised by the eukaryotic cell, as long as such mismatches do not prevent homologous recombination. Such a mismatch in the homology arms may be employed in order to introduce mutations in the nucleic acid sequence of interest and/or to avoid a target sequence of a nuclease used for inducing homologous recombination in the homology arms.

The term “heterologous recombination” as used herein refers to the integration of the nucleic acid molecule of the invention in a nucleic acid sequence comprised by the eukaryotic cell which is not homologous to the nucleotide sequence of the homology arms of the nucleic acid molecule of the invention, i.e. random integration. Therefore, heterologous recombination is not site-specific and results in an integration of the nucleic acid molecule of the invention in an undesired sequence comprised by the eukaryotic cell or off-site. The selection marker indicating heterologous recombination is not flanked by the homology arms and is thus not situated between the homology arms. Without being bound by theory, the present inventors assume that in the event of heterologous recombination the part of the nucleic acid molecule of the invention which is integrated in the nucleic acid sequence comprised by the eukaryotic cell is different compared to the part of the nucleic acid molecule of the invention which is integrated in the nucleic acid sequence comprised by the eukaryotic cell upon homologous recombination. More precisely, in case of homologous recombination it is assumed that only the nucleic acid sequence of the nucleic acid molecule of the invention comprising the homology arms and the nucleic acid sequence flanked by the homology arms (e.g. selection marker indicating homologous recombination) is integrated in the sequence of interest comprised by the eukaryotic cell, whereas in case of heterologous recombination also nucleic acid sequences of the nucleic acid molecule of the invention may be integrated in the nucleic acid sequence comprised by the eukaryotic cell which are not comprised by the nucleic acid sequence comprising the homology arms and the sequence flanked by the homology arms, e.g. the complete nucleic acid molecule of the invention. Therefore in case of heterologous recombination also the second selection marker indicating heterologous recombination may be integrated in the nucleic acid sequence comprised by the eukaryotic cell and is therefore indicative of an unwanted off-site integration or random integration event.

As described herein, the presence or absence of any one of the described selection markers is indicative of a specific genetic modification of the nucleic acid sequence comprised by the eukaryotic cell. However, the presence or absence of the described selection markers provides no guarantee for a specific genetic modification of the nucleic acid sequence comprised by the eukaryotic cell. Therefore, a further verification, e.g. sequencing, may be used for verification of the genetic modification as described herein.

The term “nucleic acid sequence of interest” as used herein refers to any nucleic acid sequence comprised by a eukaryotic cell, such as a plant or eukaryotic cell which is intended to be genetically modified.

In a preferred embodiment the nucleic acid sequence of interest comprised by said eukaryotic cells, e.g. plant or mammalian cell is in the genome of said eukaryotic cell.

The term “genome” as used herein includes the cellular genome, mitochondrial genome and/or chloroplast genome. The latter, if the eukaryotic cell is a plant cell. For eukaryotic cells, said term includes the cellular genome and/or mitochondrial genome.

In a further preferred embodiment of the invention the nucleotide sequence encoding a selection marker indicating homologous recombination when integrated in the sequence of interest and said selection marker indicating heterologous recombination when not integrated in the sequence of interest each comprises a promoter driving expression of said selection markers. Also envisioned herein is that the gene encoding the selection marker indicating homologous recombination and the gene encoding the selection marker indicating heterologous recombination are comprised in an expression cassette.

The term “promoter” as used herein is a non-coding expression control sequence preferably inserted nearby the start of the coding sequence of the expression cassette and regulates its expression. Put into a simplistic yet basically correct way, it is the interplay of the promoter with various specialized proteins called transcription factors that determine whether or not a given coding sequence may be transcribed and eventually translated into the actual protein encoded by the gene. It will be recognized by a person skilled in the art that any compatible promoter can be used for recombinant expression in host cells. The promoter itself may be preceded by an upstream activating sequence, an enhancer sequence or combination thereof. These sequences are known in the art as being any DNA sequence exhibiting a strong transcriptional activity in a cell and being derived from a gene encoding an extracellular or intracellular protein. It will also be recognized by a person skilled in the art that termination and polyadenylation sequences may suitably be derived from the same sources as the promoter. The promoter may be constitutive or inducible.

Expression cassettes as used herein contain transcriptional control elements suitable to drive transcription such as e.g. promoters, enhancers, polyadenylation signals, transcription pausing or termination signals. For proper expression of the polypeptides, suitable translational control elements are preferably included, such as e.g. 5′ untranslated regions leading to 5′ cap structures suitable for recruiting ribosomes and stop codons to terminate the translation process.

The terms “inducible” or “inducible promoter” as used herein refer to a promoter that regulate the expression of an operably linked gene in response to the presence or absence of an endogenous or exogenous stimulus. Such stimuli can be but are not limited to chemical compounds or environmental signals. This is in contrast to a constitutive promoter which does not require any stimulus to induce expression of an operably linked gene but constitutively drives the expression of said gene.

In an even further preferred embodiment of the invention the nucleotide sequence encoding a selection marker indicating homologous recombination comprises 5′ and 3′ nucleotide sequences (i.e. excision elements) which allow excision of said nucleotide sequence encoding said selection marker.

The excision elements comprised by the nucleic acid molecule of the invention preferably flank the selection marker indicating homologous recombination 5′ and 3′. Thus, in a preferred embodiment of the invention the nucleic acid sequence comprised by the nucleic acid molecule of the invention comprising the selection marker indicating homologous recombination is arranged as follows:

homology arm-excision element-selection marker indicating homologous recombination operably linked to a promoter-poly A-excision element-homology arm.

In case two of said nucleic acid molecules comprising a different nucleotide sequence encoding a selection marker indicating homologous recombination are used, one or both alleles can be targeted, e.g. in order to introduce a point mutation. Subsequently, the selection markers may be excised. By way of example, such a genetically modified cell may be a suitable model system for a disease allowing to test and identify novel therapeutic agents.

In a preferred embodiment the nucleotide sequences which allow excision of the nucleotide sequence encoding the selection marker indicating homologous recombination are selected from loxP sequences, derivatives of loxP sequences, such as Lox511, Lox5171, Lox2272, M2, M3, M7, M11, Lox71 or Lox66 sequences, Cre recombinase binding sites, FRT sequences, derivatives of FRT sequences, such as FRT-G, FRT-H or FRT-F3, FLP recombinase binding sites, terminal repeats, transposase binding sites, derivatives of transposase binding sites, such as terminal repeats, internal terminal repeats, direct repeats, inverted repeats or palindromic repeats, piggybac transposon binding sites, sleeping beauty transposon binding sites, piggybat binding sites, binding site of transposons fused to estrogen receptor, estrogen binding sites or mutational derivatives, binding site of transposons fused to estrogen receptor tamoxifen binding sites, RNA-guided nuclease binding site, Cas9 binding sites, Cpf binding site, nuclease binding sites, transcription activator like effector nuclease (TALEN or TALEN-Fok1) binding sites, zinc finger nuclease (ZFN) binding sites or RNA-guided nuclease binding sites.

The nucleotide sequence encoding a selection marker indicating homologous recombination may be removed by excision or recombination or cleavage after depositing a modification into the genome. The removal of the nucleotide sequence encoding a selection marker indicating homologous recombination may be performed by the use of a recombinase, transposase, RNA guided nuclease or nuclease. The excision, recombination or cleavage of the nucleotide sequence encoding a selection marker indicating homologous recombination may be induced by the expression of a recombinase, transposase, RNA guided nuclease or nuclease. Such enzyme can be delivered by electroporation or transfection of a double stranded DNA, transfection of a pre transcribed mRNA or pre translated protein. When such enzyme is fused to a nuclear receptor domain (e.g. estrogen receptor) it can be translocated into the nucleus by the supplementation of tamoxifen, to induce ER domain (or nuclear receptor translocation domains) mediated nuclear translocation. Such enzyme may also be induced by the supplementation of doxycycline to the media, inducing the tetracycline-controlled transcriptional activation of its gene. Cells that have undergone removal of the nucleotide sequence encoding a selection marker indicating homologous recombination may further be enriched by the selection of cells that lost the fluorescence signal by FACS or fluorescence microscopy. After the removal of the positive selection module homozygous gene corrected edited lines, homozygous mutant genome edited lines, heterozygous mutant genome edited lines or heterozygous gene corrected edited lines can be subcloned or used for phenotypic characterization, drug screening or cell therapy.

In a further preferred embodiment the nucleic acid molecule of the invention comprises a chemical resistance selection marker selected from neomycin resistance, hygromycin resistance, HPRT1, puromycin resistance, puromycin N-acetyl-transferase, blasticidin resistance, G418 resistance, phleomycin resistance, nourseothricin resistance or chloramphenicol resistance, puromycin resistance being preferred.

It is envisioned herein that the chemical resistance selection marker is associated or not associated with the selection marker indicating homologous recombination. Accordingly, the chemical resistance selection marker may be associated with the selection marker indicating homologous recombination via a linking element, e.g. P2A, T2A, E2A, F2A or and internal ribosome entry site (IRES), T2A being preferred. Thus, in a preferred embodiment of the invention the genetic element comprised by the nucleic acid molecule of the invention comprising the selection marker indicating homologous recombination is arranged as follows:

homology arm-excision element-selection marker indicating homologous recombination operably linked to a promoter-linking element-chemical resistance marker-poly A-excision element-homology arm.

In a preferred embodiment of the invention the nucleotide sequences that are homologous (homology arms) to nucleotide sequences of a nucleic acid sequence of interest comprised by said eukaryotic cell allow homologous recombination with nucleotide sequences of a nucleic acid sequence of interest comprised by said eukaryotic cell. However, as described elsewhere herein the homology arms may comprise one or more mismatches compared to the nucleic acid sequence of interest comprised by the eukaryotic cell, as long as such mismatches do not prevent homologous recombination. Such a mismatch in the homology arms may be employed in order to introduce mutations in the nucleic acid sequence of interest.

Accordingly, in a preferred embodiment the present invention employs homologous recombination for depositing a modification into the genome, said modification is selected from a single nucleotide polymorphism (SNP), phosphomimetic mutation, phospho null mutation, missense mutation, nonsense mutation, synonymous mutation, insertion, deletion, knock-out or knock-in.

A preferred SNP that may be introduced into the alpha synuclein gene or an othologue thereof of a eukaryotic cell, preferably of a human induced pluripotent stem cell is one which results in the following mutation A30P, A53T, E46K, G51D or H50Q. Of course, any preferred SNP can be introduced in any preferred gene of any eukaryotic cell, since techniques for introducing SNPs or knocking-in or knocking-out genes by means and methods facilitating homologous recombination are well known in the art and exemplarily described herein, such as CRISPR/Cas.

The modification may be deposited in the genome of the cell preferably by a mismatch in the homology arms of the nucleic acid molecule, which is integrated in the genome of the cell upon homologous recombination, compared to the homologous nucleotide sequences of a nucleic acid sequence of interest. However, a modification may also be deposited by inserting a nucleic acid sequence or modification to be deposited in the genome of the cell between the homology arms in the nucleic acid molecule of the invention.

In a preferred embodiment the homologous recombination occurs at one allele (mono allelic) or at both alleles (bi-allelic) of said nucleic acid sequence of interest comprised by said eukaryotic cell.

In a further preferred embodiment homologous recombination is mediated by TALENs, ZFNs, meganucleases, or CRISPR/Cas. Homologous recombination of the nucleic acid molecule of the invention with the nucleic acid sequence of interest comprised by said eukaryotic cell is a rare event. It is known in the art that inducing a DNA double-strand break in nucleic acid sequences comprised by a cell induces cellular DNA repair mechanisms, such as homologous recombination. Thus, the induction of a site specific DNA double-strand break in the nucleic acid sequence of interest increases homologous recombination with the nucleic acid molecule of the invention, being as well directed to the nucleic acid sequence of interest via the homology arms. Such a DNA double-strand break may be induced by employing TALENs, ZFNs, meganucleases, or CRISPR/Cas, or any other nuclease being directed to the nucleic acid sequence of interest comprised by the eukaryotic cell.

The term “TALEN” as used herein refers to transcription activator-like effector nucleases which are fusions of the Fokl cleavage domain and DNA-binding domains derived from TALE proteins. As the Fokl cleavage domain is only active as a dimer, two TALENs have to be used which bring the Fokl cleavage domains in close proximity upon binding to their target sequence resulting in the induction of a DNA double-strand break. TALEs contain multiple 33-35-amino-acid repeat domains that each recognizes a single base pair with the so-called repeat variable di-residue (RVD) which are two variable amino acids determining the binding specificity one single repeat. Like ZFNs, TALENs induce targeted DSBs that activate DNA damage response pathways and enable custom alterations. However, TALENs may also be modified such that one of the Fokl cleavage domains is inactivated. Such modified TALENs cause only single-strand breaks and are thus nickases.

The term “ZFN” or “zinc-finger nuclease” as used herein refers to fusions of the nonspecific DNA cleavage domain from the Fokl restriction endonuclease with zinc-finger proteins. ZFN dimers induce targeted DNA DSBs that stimulate DNA damage response pathways. The binding specificity of the designed zinc-finger domain directs the ZFN to a specific genomic site. However, also zinc-finger nickases (ZFNickases) may be used. Zinc-finger nickases are ZFNs that contain inactivating mutations in one of the two Fokl cleavage domains. ZFNickases make only single-strand DNA breaks and induce HDR without activating the mutagenic NHEJ pathway.

The term “meganuclease” as used herein refers to a family of endonucleases, also called homing endonucleases that can be divided into five families based on sequence and structure motifs: LAGLIDADG, GIY-YIG, HNH, His-Cys box and PD-(D/E)XK characterized by a large recognition site (double-stranded DNA sequences of 12 to 40 base pairs). The most well studied family is that of the LAGLIDADG proteins, including I-SceI, I-CreI and I-DmoI which are most widely used in research and genome engineering. The best known LAGLIDADG endonucleases are homodimers (I-CreI) or internally symmetrical monomers (I-SceI). The DNA binding site, which contains the catalytic domain, is composed of two parts on either side of the cutting point. The half-binding sites can be extremely similar and bind to a palindromic or semi-palindromic DNA sequence (I-CreI), or they can be non-palindromic (I-SceI).

The term “CRISPR/Cas” as used herein relates to the Clustered Regularly Interspaced Short Palindromic Repeats (CRISPR) Type II system which is a bacterial immune system that has been modified for genome engineering. CRISPR consists of two components: a “guide” RNA (gRNA) and a non-specific CRISPR-associated endonuclease (Cas9). The gRNA is a short synthetic RNA composed of a “scaffold” sequence necessary for Cas9-binding and a user-defined ˜20 nucleotide “spacer” or “targeting” sequence which defines the genomic target to be modified. Thus, one can change the genomic target of Cas9 by simply changing the targeting sequence present in the gRNA. CRISPR/Cas can be used for gene engineering by co-expressing a gRNA specific to the sequence to be targeted and the endonuclease Cas9. The genomic target can be any ˜20 nucleotide DNA sequence, provided the sequence is unique compared to the rest of the genome and the target is present immediately upstream of a Protospacer Adjacent Motif (PAM). The PAM sequence is absolutely necessary for target binding and the exact sequence is dependent upon the species of Cas9.

In a further preferred embodiment of the invention the nucleotide sequences that are homologous (homology arms) to nucleotide sequences of a nucleic acid sequence of interest comprised by the eukaryotic cell do not comprise a target sequence for TALENs, ZFNs, meganucleases, or CRISPR/Cas or any other nuclease which mediate homologous recombination.

As nucleotide sequences that are homologous (homology arms) to nucleotide sequences of a nucleic acid sequence of interest are intended to be integrated in the nucleic acid sequence of interest comprised by the eukaryotic cell or the cellular genome, cleavage by the nuclease used to mediate homologous recombination is to be avoided. Thus, it is envisioned that the homology arms do not comprise a target sequence for a nuclease used to mediate homologous recombination. In order to avoid a target sequence within the homology arms the nucleic acid sequence of the homology arms may be modified such that the nuclease does not recognize it as a target sequence or the binding affinity of the nuclease is reduced. Thus the nuclease used to mediate homologous recombination can only induce a DNA double-strand break in the nucleotide sequence of interest comprised by the cell but not in the integrated nucleic acid molecule of the invention or the non-integrated nucleic acid molecule of the invention comprised by the eukaryotic cell. However, the target sequence of the nuclease may also be splitted in the nucleic acid molecule of the invention by the selection marker indicating homologous recombination such that the nuclease cannot recognize it as a target sequence or the binding affinity of the nuclease is reduced. Without being bound by theory, such an unwanted DNA double-strand break could induce the error prone DNA repair mechanism non-homologous end joining and would thus introduce unwanted mutations in the nucleic acid molecule of the invention or in the nucleic acid molecule of the invention integrated in the cellular genome. Accordingly, using two nucleic acid molecules comprising different nucleotide sequences encoding different selection markers advantageously indicate that two homologous recombination events have occurred in cells being double positive for both markers and consequently no unwanted mutations have been introduced via non-homologous end joining in any one of the targeted alleles.

The term “non-homologous end joining” as used herein refers to a DNA repair pathway that ligates or joins two broken ends together. NHEJ does not use a homologous template for repair and thus typically leads to the introduction of small insertions and deletions at the site of the break, often inducing frame-shifts that knockout gene function.

In another preferred embodiment the nucleic acid molecule of the invention is a vector.

The term “vector” as used herein refers to a nucleic acid molecule into which the nucleic acid molecule of the invention may be inserted or cloned. The vector may encodes a further antibiotic resistance gene. The vector may be an expression vector. The vector may be capable of autonomous replication in a host cell (e. g., vectors having an origin of replication which functions in the host cell). The vector may have a linear, circular, or supercoiled configuration and may be complexed with other vectors or other material for certain purposes.

One type of vector is a plasmid, which refers to a circular double stranded DNA loop into which additional DNA segments may be introduced via ligation or by means of restriction-free cloning. Other vectors include cosmids, bacterial artificial chromosomes (BAC), yeast artificial chromosomes (YAC) or mini-chromosomes. Another type of vector is a viral vector, wherein additional DNA segments may be ligated into the viral genome.

In a preferred embodiment of the invention the vector is circular or linearized.

In yet another preferred embodiment of the invention the optical discriminability is different emission wavelength, e.g. different emission wavelength in the fluorescence range.

The term “optically discriminable” as used herein preferably relates to a difference in emission wavelength, e.g. different colors such that the selection marker indicating homologous recombination and the selection marker indicating heterologous recombination can be distinguished upon detection, e.g. using FACS, fluorescence microscopy or flow cytometry.

In a preferred embodiment of the invention the selection marker indicating homologous recombination and the selection marker indicating heterologous recombination is a fluorescent protein. The fluorescent protein is preferably selected from Sirius, SBFP2, Azurite, EBFP2, mKalama1, mTagBFP2, Aquamarine, ECFP, Cerulean, mCerulean3, SCFP3A, mTurquoise2, CyPet, AmCyan1, mTFP1, MiCy, iLOV, AcGFP1, sfGFP, mEmerald, EGFP, mAzamiGreen, cfSGFP2, ZsGreen, mWasabi, SGFP2, Clover, mClover2, EYFP, mTopaz, mVenus, SYFP2, mCitrine, YPet, ZsYellow1, mPapaya1, mKO, mOrange, mOrange2, mKO2, TurboRFP, mRuby2, eqFP611, DsRed2, mApple, mStrawberry, FusionRed, mRFP1, mCherry, mCherry2, dTOMATO, tdTOMATO, tagBFP, photoactivatable or photoswitchable fluorescent protein.

The fluorescent proteins applied in the different nucleic acid molecules of the present invention are advantageously chosen such that they are optically discriminable from each other. A pair of fluorescent proteins applied in connection with the selection marker indicating homologous recombination is optically discriminable from each other and is optically discriminable from the fluorescent protein applied in connection with the selection marker indicating heterologous recombination.

Exemplary combinations of fluorescent proteins are as follows:

| | selection | selection |
|---|---|---|
| | marker | marker |
| selection | indicating | indicating |
| marker | homologous | homologous |
| indicating | recombination | recombination |
| heterologous | (nucleic acid | (nucleic acid |
| recombination | molecule 1) | molecule 2) |
| tagBFP | EGFP | dTOMATO |
| tagBFP | EGFP | EBFP2 |
| tagBFP | EGFP | Cerulean |
| tagBFP | EGFP | SYFP2 |
| tagBFP | EGFP | mRFP1 |
| EGFP | tagBFP | dTOMATO |
| dTOMATO | EGFP | tagBFP |

In another preferred embodiment the invention provides a composition of matter comprising a mixture of at least two different nucleic acid molecules of the invention, each comprising a different nucleotide sequence encoding a selection marker indicating homologous recombination in said eukaryotic cell.

Such a composition of matter, comprises different nucleic acid molecules of the invention, differing in the nucleotide sequence encoding a selection marker indicating homologous recombination in the eukaryotic cell such that the selection markers when being expressed are optically discriminable as described herein. Such a composition of matter is advantageous in detecting cells in which both alleles have been successfully genetically modified as the presence of both selection markers in the eukaryotic cell are indicative of two homologous recombination events and thus genetic modification of both alleles. By way of example, one nucleic acid molecule comprises a selection marker gene indicating homologous recombination encoding for EGFP, whereas the second nucleic acid molecule comprises a selection marker gene indicating homologous recombination encoding for dTomato. Thus, without being bound by theory a cell being positive for EGFP and dTomato (and being negative for the selection marker indicating heterologous recombination) has two site specific integrations of the nucleic acid molecules of the invention and has therefore the genetic modification in both alleles. Such a cell may be positive for EGFP and dTomato or may also be yellow, due to the presence of green and red, when subjected to FACS analysis or fluorescence microscopy. The discrimination between the two nucleic acid molecules differing in the nucleotide sequence encoding a selection marker indicating homologous recombination in the eukaryotic cell further allows to detect cells in which both alleles have different genetic modifications. By way of example, the first nucleic acid molecules may comprise a wild type sequence and an EGFP selection marker indicating homologous recombination, whereas the second nucleic acid molecules may comprise a pathologic mutation and a dTomato selection marker indicating homologous recombination. Thus, a cell being EGFP and dTomato positive (or yellow) when subjected to FACS analysis or fluorescence microscopy has both nucleic acid molecules integrated and is thus heterozygous with respect to the genetically modified sequence of interest. However, it is also envisioned to discriminate cells which are only positive for a single selection marker by way of the expression level of the selection marker gene, e.g. brightness of the fluorescence due to the expression of a fluorescent protein. Without being bound by theory, it is expected that cells having two integrations of the nucleic acid molecule of the invention encoding e.g. a fluorescent protein are brighter when subjected to FACS analysis or fluorescence microscopy and thus have both alleles genetically modified.

In a further preferred embodiment the invention provides an in vitro method for enriching eukaryotic cells which are modified by homologous recombination, comprising
(a) subjecting a population of cells transformed with a nucleic acid molecule or a composition of the invention to means for selecting for said marker indicating heterologous recombination and separate transformed cells expressing said selection marker indicating heterologous recombination in said eukaryotic cell; and
(b) subjecting the non-separated cells to means for selecting for said marker indicating homologous recombination in order to enrich transformed cells comprising said homologous recombination.

The in vitro methods of the invention make use of the discriminable selection markers by separating in a first step (a) cells which comprise the selection marker indicating heterologous recombination and therefore an unspecific and/or off-site integration of the nucleic acid molecule or the composition of the invention in the eukaryotic cell. In the second selection step (b) non-separated cells (cells which are not positive for the selection marker indicating heterologous recombination) are selected for said marker indicating homologous recombination indicating a site-specific integration of the nucleic acid molecule or the composition of the invention. Said second selection step separates cells which do not comprise the selection marker, indicating that said cells were not successfully transformed. In case a composition of the invention is used in the in vitro methods of the invention two different selection markers may be detected, as the nucleic acid molecules of the composition differ in the nucleotide sequence encoding a selection marker indicating homologous recombination. Thus, in case nucleic acid molecule one comprises marker 1 and nucleic acid molecule two comprises marker 2 a cell may comprise the following combinations of selection markers with respect to two alleles of the nucleic acid sequence of interest: marker 1, marker 2, marker 1 and marker 1, marker 2 and marker 2, and marker 1 and marker 2 (or marker 2 and marker 1). Said combinations of selection markers provide further information on the genetic modification of the cell. In case a cell is single positive for marker 1 or marker 2 this indicates a single integration of one nucleic acid molecule of the invention and therefor the modification of one allele, only. In case a cell is double positive for marker 1 or marker 2 (i.e. marker 1 and marker 1 or marker 2 and marker 2) this indicates two integrations of the same nucleic acid molecule of the invention. Thus, both alleles of the cell are likely to be modified and the cell is likely to be homozygous with respect to the modified allele (sequence of interest). Without being bound by theory, such a cell appears more positive when subjected to detection means suitable to detect the marker (e.g. brighter in color in case of fluorescent proteins). In case a cell is positive for marker 1 and marker 2 this indicates two integrations (one integration of each nucleic acid molecule of the invention). Thus, both alleles of the cell are likely to be modified. As the combination of two different markers, which are discriminable, preferably optical discriminable, such as different fluorescent proteins differing in emission wavelength, are easily detectable by a person skilled in the art, such a cell is preferred. In case the two different nucleic acid molecules of the invention further differ in the genetic modification introduced upon homologous recombination in the eukaryotic cell, such a cell would be likely heterozygous with respect to the modified allele (sequence of interest).

By way of example, FIG. 2 shows possible outcomes for nucleic acid molecules with different combinations of fluorescent markers and homology arms with or without mismatches. The upper panel shows a possible scenario in which the fluorescent marker EGFP is combined with homology arms without mismatches in the first nucleic acid molecule and the fluorescent marker dTomato is combined with homology arms with a single mismatch in the second nucleic acid molecule. Consequently, a target cell being double positive of EGFP and d Tomato will be heterozygous with one wild type allele and one mutated allele comprising a SNP. The middle panel shows a possible scenario in which the fluorescent marker EGFP is combined with homology arms without mismatches in the first nucleic acid molecule and the fluorescent marker dTomato is also combined with homology arms without mismatches in the second nucleic acid molecule. Consequently, a target cell being double positive of EGFP and dTomato will be homozygous with two wild type alleles. The lower panel shows a possible scenario in which the fluorescent marker EGFP is combined with homology arms with a single mismatch in the first nucleic acid molecule and the fluorescent marker dTomato is also combined with homology arms with a single mismatch in the second nucleic acid molecule. Consequently, a target cell being double positive of EGFP and d Tomato will be homozygous with two mutated alleles comprising a SNP.

The above described single and double integrations of one marker also applies mutatis mutandis to the use of one nucleic acid molecule of the invention. However, in case the nucleic acid molecule or the nucleic acid molecules comprised by the composition of matter further comprise a chemical resistance marker, the cells may be subjected to chemical selection prior to subjecting to selection for the marker indicating heterologous recombination and the marker indicating homologous recombination.

The selection process described for the in vitro methods of the invention may also be performed more than one time, such as 2, 3, 4, 5, 6, 7, 8, 9 or more times to further enrich the eukaryotic cell comprising the desired homologous recombination or mutation.

The term “enriching” as used herein also refers to “selecting” and “obtaining” eukaryotic cells which are modified by homologous recombination.

The term “means for selecting” as used herein refers to means which are suitable to detect the selectable marker. In case of an optically detectable marker, e.g. a fluorescent protein, such means may be but are not limited to FACS, fluorescent guided capture, e.g. colony/clone picking by detecting fluorescent colonies/clones, flow cytometry or fluorescence microscopy.

The term “transformed” or “transformation” as use herein refers to any method suitable to deliver or transport nucleic acid to the cell, such as transformation, transfection, transduction, electroporation, magnetofection, lipofection and the like, electroporation being preferred.

In a further preferred embodiment of the invention the in vitro method for enriching eukaryotic cells which are modified by homologous recombination further comprises (c) subjecting said enriched cells to sequencing.

The term “sequencing” as used herein refers to obtaining sequence information from a nucleic acid strand, typically by determining the identity of at least some nucleotides (including their nucleobase components) within the nucleic acid molecule. While in some embodiments, “sequencing” a given region of a nucleic acid molecule includes identifying each and every nucleotide within the region that is sequenced, in some embodiments “sequencing” comprises methods whereby the identity of only some of the nucleotides in the region is determined, while the identity of some nucleotides remains undetermined or incorrectly determined. “Sequencing” may refer to obtaining sequence information of a region or of the whole genome of the cell. Any suitable method of sequencing may be used, such as label-free or ion based sequencing methods, labeled or dye-containing nucleotide or fluorescent based nucleotide sequencing methods, or cluster-based sequencing or bridge sequencing methods.

In a further preferred embodiment of the invention the in vitro method for enriching eukaryotic cells which are modified by homologous recombination further comprises removing the nucleotide sequence encoding said marker indicating homologous recombination. Said marker indicating homologous recombination is preferably removed by excision as described herein.

In another preferred embodiment the invention provides an in vitro method for producing eukaryotic cells comprising a modification in its genome introduced by homologous recombination, comprising
(a) subjecting a population of cells transformed with a nucleic acid molecule of the invention or a composition of the invention to means for selecting for said marker indicating heterologous recombination and separate transformed cells expressing said selection marker indicating heterologous recombination in said eukaryotic cell;
(b) subjecting the non-separated cells to means for selecting for said marker indicating homologous recombination in order to enrich transformed cells comprising said homologous recombination; and
(c) sequencing said enriched transformed cells in order to determine whether said enriched transformed cells comprise the desired modification.

In a further preferred embodiment of the invention the in vitro method for producing eukaryotic cells comprising a modification in its genome introduced by homologous recombination further comprises removing the nucleotide sequence encoding said marker indicating homologous recombination. Said marker indicating homologous recombination is preferably removed by excision as described herein.

The present invention also provides the use of a nucleic acid molecule as described herein or a composition as described herein for enriching eukaryotic cells which are modified by homologous recombination.

Furthermore, the present invention provides the use of a nucleic acid as described herein or a composition as described herein for producing eukaryotic cells comprising a modification in its genome introduced by homologous recombination.

The present invention allows to discriminate between different nucleic acid molecules of the invention, due to different selection markers, wherein the nucleic acid molecules target the same sequence of interest. The different nucleic acid molecules may further deposit different modifications into the genome of the cell. Thus a cell being positive for both markers is likely to be heterozygous with respect to the sequence of interest. Such a cell may comprise a mutation in one allele, whereas the other allele comprises the wild type nucleic acid sequence giving rise to bi-allelically targeted homozygotes and heterozygote cell populations at will. This allows the generation of cell lines which are homozygous gene corrected edited lines, homozygous mutant genome edited lines, heterozygous mutant genome edited lines or heterozygous gene corrected edited lines which may be subcloned or used for phenotypic characterization, drug screening or cell therapy.

### FIGURES

FIG. 1: Examples of nucleic acid molecules of the invention and selection process.

A) Representation of a standard donor vector containing a fluorescent positive selection module expressing EGFP or dTOMATO and a negative selection module expressing tagBFP

B) Expected outcomes for the derived population as designed with each dsDNA donor.

C) Workflow of Fluorescence Assisted Genome Editing (FACE). Is shown the gating structure to exclude random integration tagBFP positive cells and include herozygotes-homozygotes combinations for each modification.

D) One specific genotype is sorted based on the fluorescence combination. Representative heterozygous EGFP and dTOMATO positive single cell and colonies after sorting.

E) The selected population is expanded and transfected with transposase to remove the positive selection module restoring the native structure of the locus. The polyclonal populations is sequenced optionally subcloned.

FIG. 2: Known outcomes of biallelic targeting.

Possible outcomes according to different combinations of fluorescent markers and homology arms with or without mismatches (this is without considering the silent mismatches introduced for avoiding the recognition of the nuclease). 1A) The fluorescent marker EGFP is combined with homology arms without mismatches and the fluorescent marker dTomato is combined with homology arms with a single mismatch, composition necessary for producing an heterozygous knock in of a desired mutation. 1B) The fluorescent marker EGFP is combined with homology arms without mismatches and the fluorescent marker dTomato is combined with homology arms without mismatches as well, composition necessary for producing an homozygous knock in with two wild type alleles. 1C) The fluorescent marker EGFP is combined with homology arms with a desired mismatch and the fluorescent marker dTomato is combined with homology arms with a desired mismatch as well, composition necessary for producing an homozygous knock in with two mutated alleles comprising a SNP.

FIG. 3: Further examples of nucleic acid molecules of the invention and selection process.

A) Representation of a standard donor vector containing a fluorescent positive selection module expressing EGFP or dTOMATO and a negative selection module expressing tagBFP

B) Expected outcomes for the derived population as designed with each dsDNA donor (in this case represented with a composition to produce a homozygous modification)

C) Workflow of Fluorescence Assisted Genome Editing (FACE).

D) Gating structure to exclude random integration tagBFP positive cells and include herozygotes-homozygotes combinations for each modification.

E) One specific genotype is sorted based on the fluorescence combination. Representative heterozygous EGFP and dTOMATO positive single cell and colonies after sorting.

F) Purification step to obtain an entire population with the desired genotype, with the presence of both fluorescences

G) The selected population is expanded and transfected with transposase to remove the positive selection module restoring the native structure of the locus.

### EXAMPLES

The following Examples illustrate the invention, but are not to be construed as limiting the scope of the invention.

### Example 1: Designing of the Donor Constructs

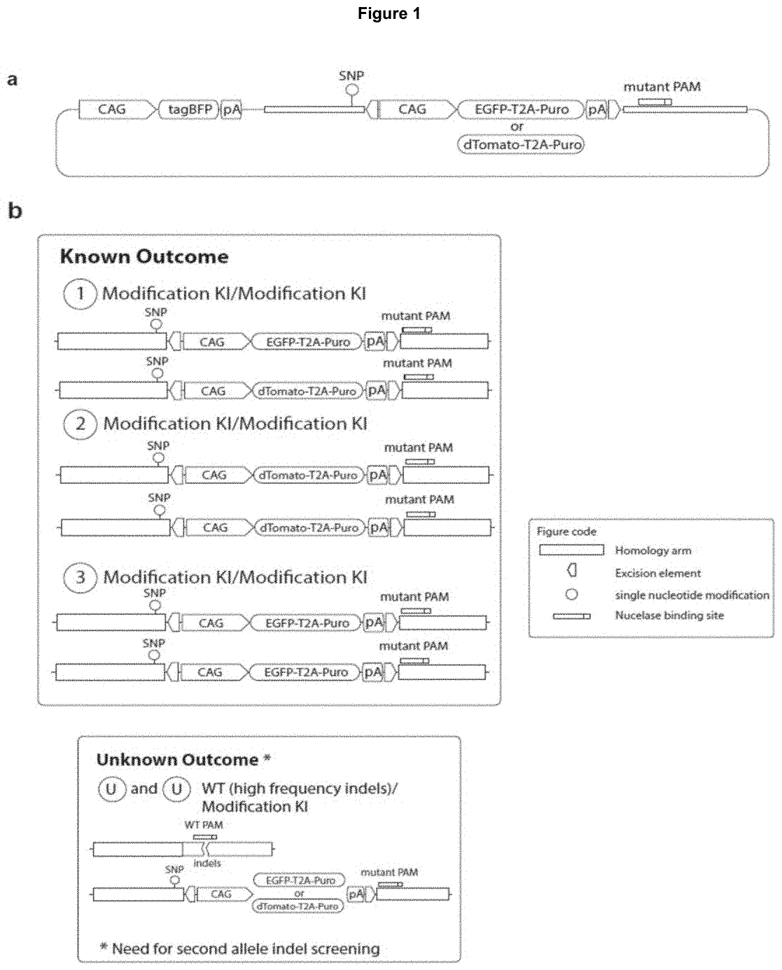

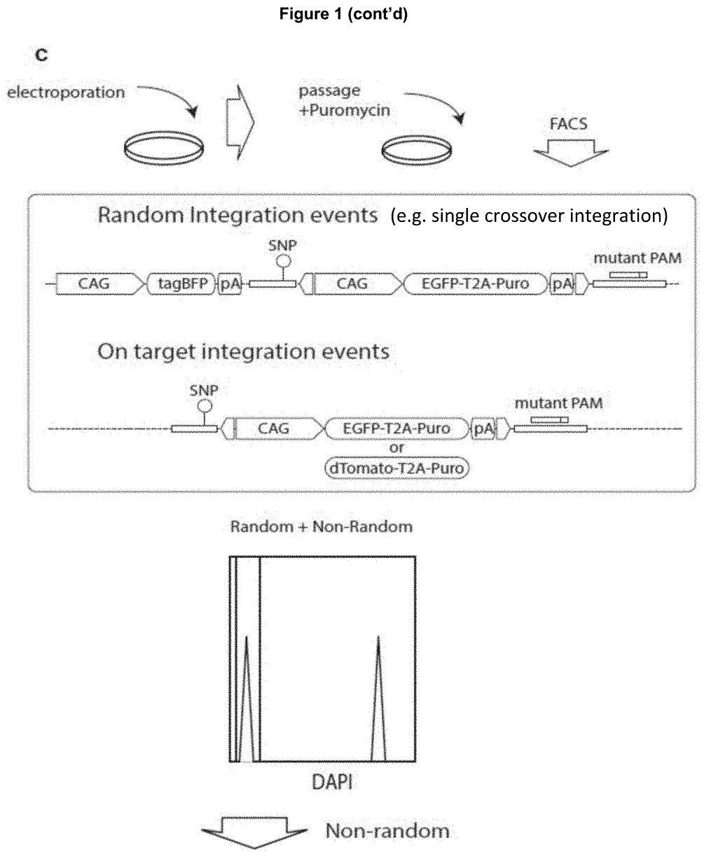

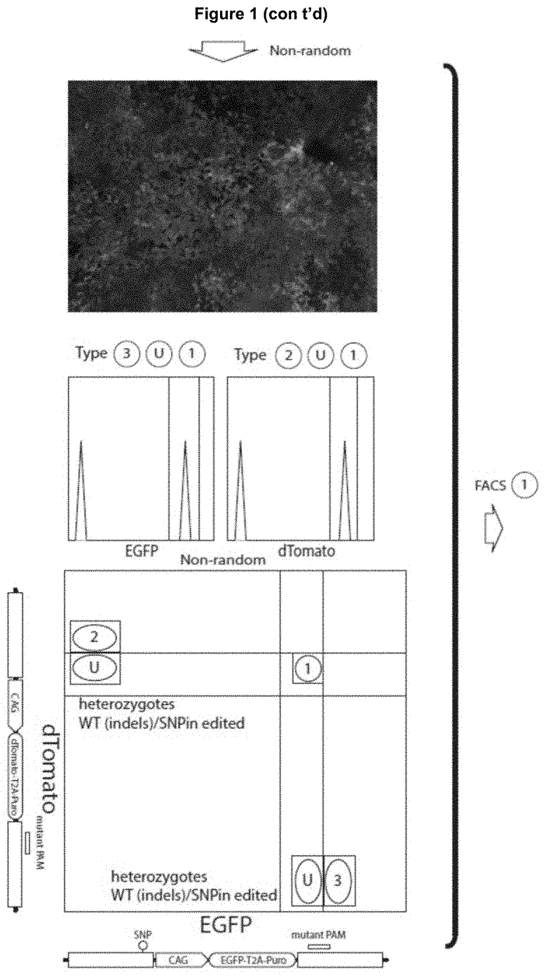

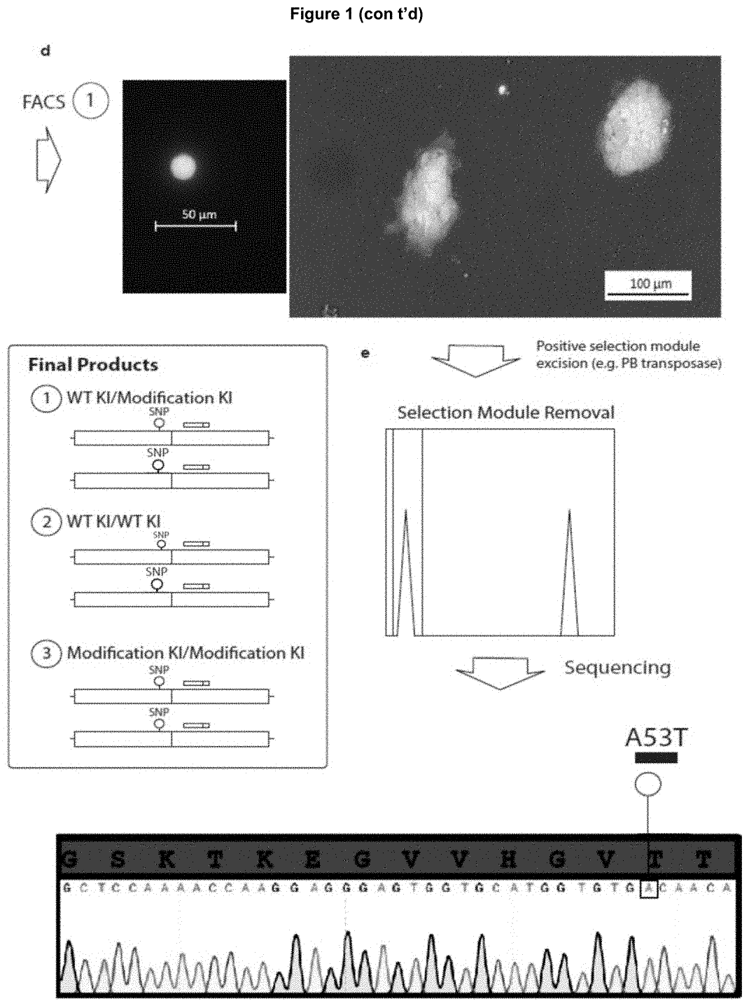

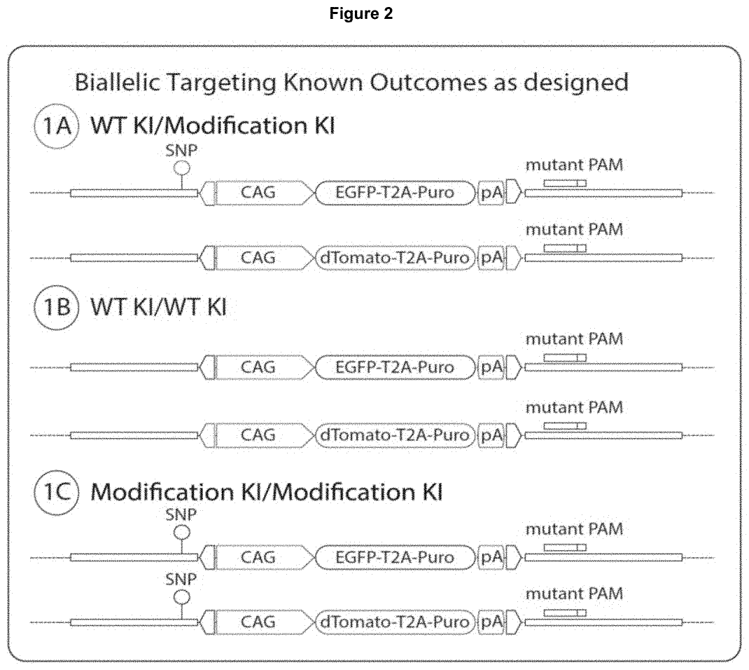

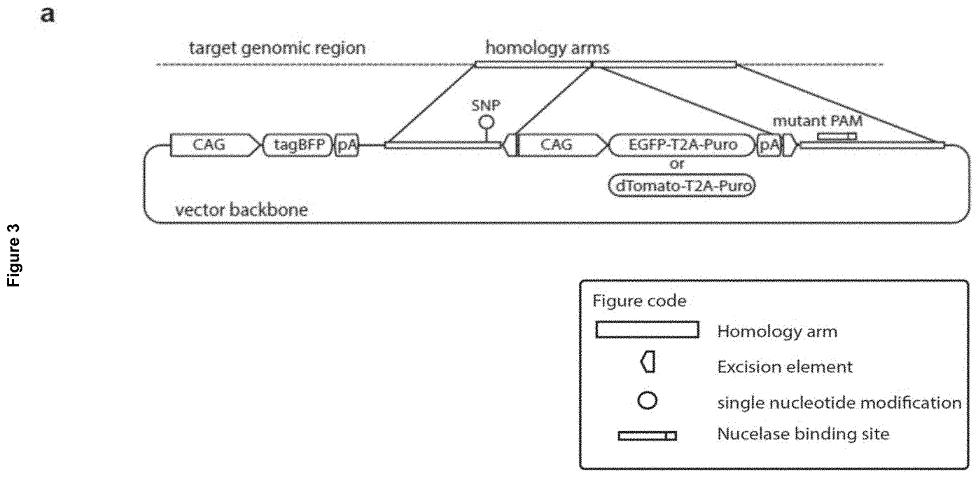

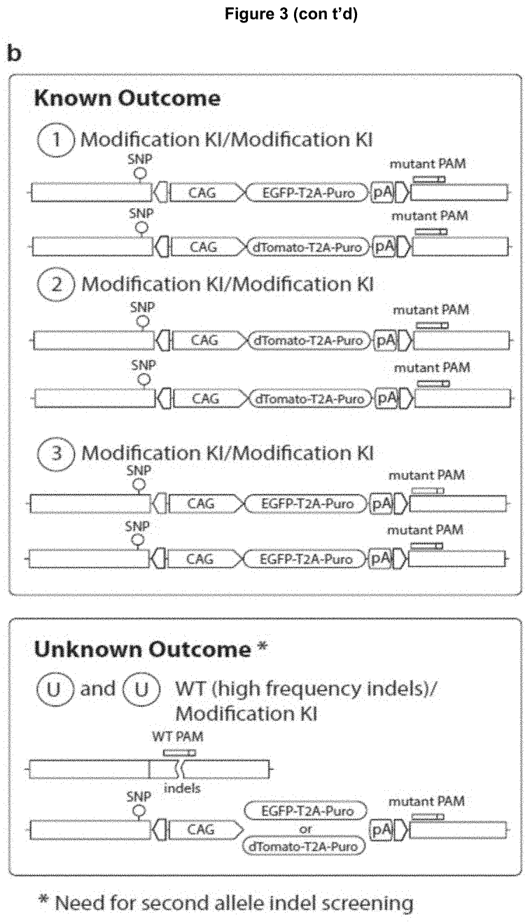

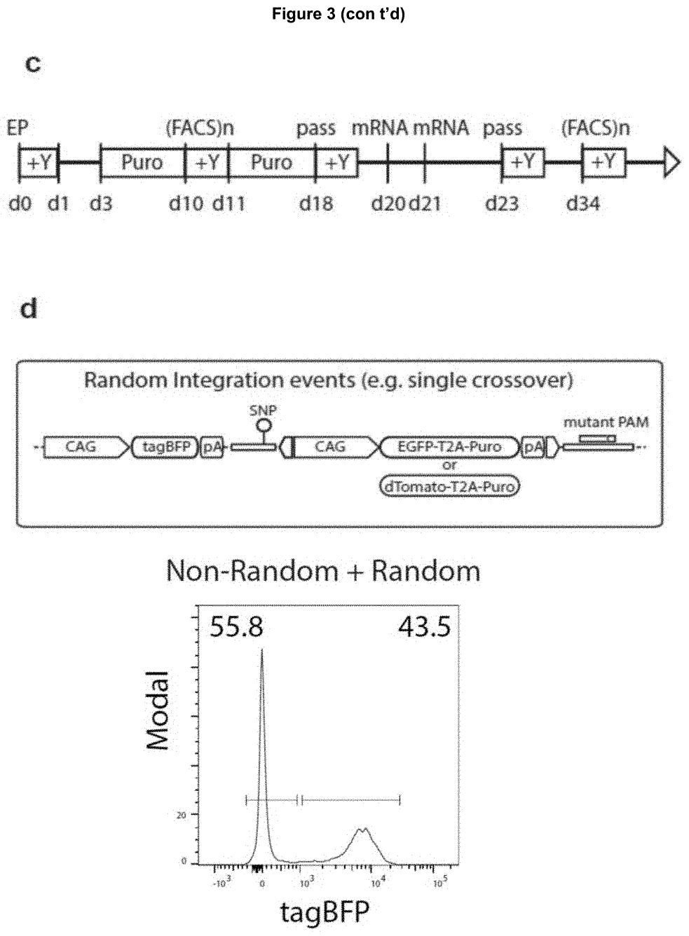

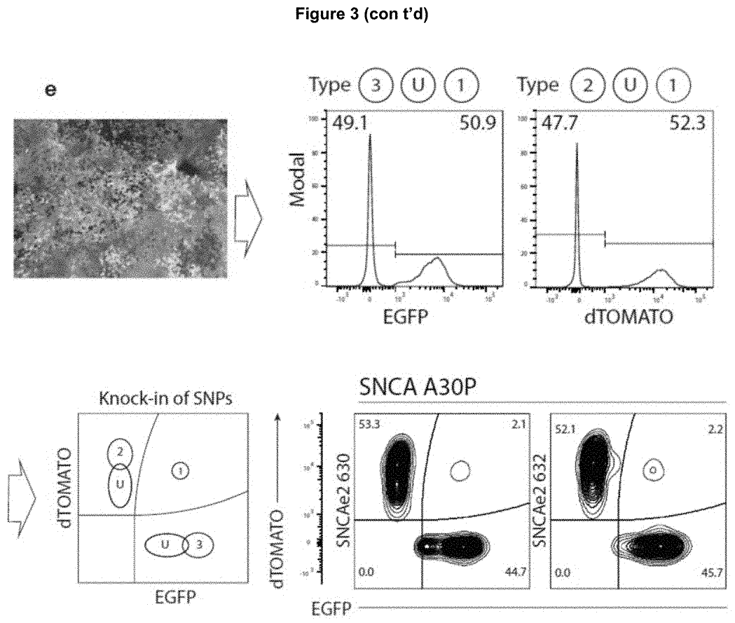

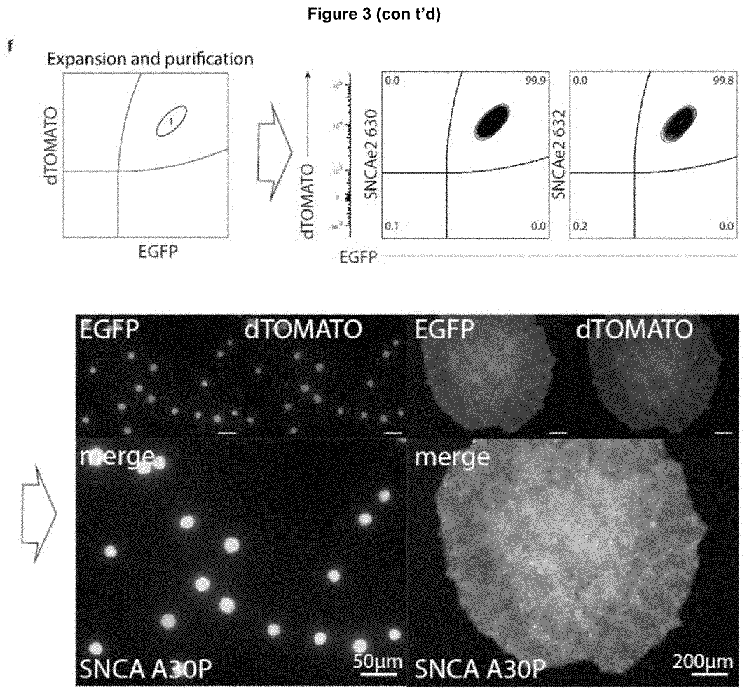

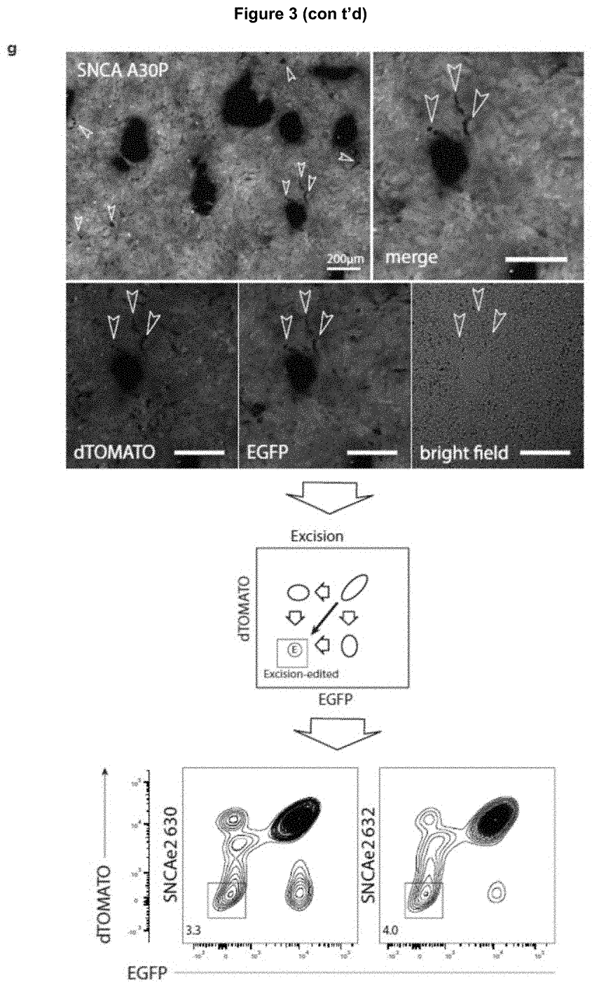

Desired nucleotide modifications are added into the homology arms targeting a specific sequence in the genome by site directed mutagenesis. When used CRISPR-Cas9 a point mutation in the PAM sequence is introduced. The homology arms are cloned into the donor scaffold by Gibson assembly as shown in FIGS. 1A and 3A. Two donor vectors are created to introduce a biallelic knock in of the donor DNA and giving rise to homozygote or heterozygote edited cells as described in FIGS. 1B and 3B. A fluorescent reporter (distinct from the one used in the positive selection cassette) is inserted externally to the homology arms and the positive selection cassette to exclude cells that present random integration events as shown in FIGS. 1A and 1C and FIGS. 3A and 3D.

### Example 2: Cell Culture Conditions and Transfection

Human induced pluripotent stem cell lines derived by episomal, mRNA or retroviral methods were used. Lines were cultured on Essential 8 media (Life Technologies) on laminin 521 or 511. For electroporation and normal passage cell lines are detached with Acutase (Life Technologies). Human induced pluripotent stem cell lines were transformed with both donor vectors and nuclease coding vector with an Amaxa nucleofector 4D kit (Lonza) and plated in media containing Rho Kinase inhibitor.

### Example 3: Enrichment of on Target Edited Cells

After electroporation cells are cultured on 0.5 ug/mL of puromicyn and expanded for up to 3 days and sorted in BD ARIAII FACS. Random integration events are evidenced by the expression of the negative selection reporter tagBFP and are discarded (FIG. 1C and FIG. 3D). Based on the fluorescent pattern, only the population presenting biallelic integration of both positive selection modules is selected by FACS or microscopy guided capture of the cells (FIGS. 1D and 1E and FIGS. 3E and 3F).

### Example 4: Removal of the Positive Selection Cassette and QC

Homogeneous biallelic populations coexpressing both positive selection module reporters are transfected with IVT mRNA (Applied Biosystems) coding codon optimized hyper transposase using Stemfect (Stemgent) according to manufacturer instruction. Two days after transfection the cells are sorted in BD ARIAII for the removal of the fluorescent reporters indicative of the excision of the positive selection module and restoration of the endogenous locus. The quality of homogenously edited polyclonal population is assessed by MiSeq deep sequencing including the analysis of nuclease off targets and random integration.

### Example 5: Knock in of Parkinson Disease Associated SNPs Using FAGE

Parkinson disease is a multifactorial neurodegenerative disorder with a limited number of mendelian linked genetic variants. Parkinson patients with mutations in the alpha synuclein gene carry the heterozygote mutations A30P, A53T, E46K, G51D or H50Q. Fast enrichment of the desired populations was achieved by the use of two donors containing the fluorescent protein EGFP or dTOMATO and a drug selection resistant gene (FIG. 1A and FIG. 3A). In order to load the genotypic combinations into wild type human induced pluripotent stem cell (hiPSC) lines for SNCA (FIG. 1B and FIG. 3B) dsDNA donors were used. In order to enhance the homology directed repair (HDR) efficiency and decrease the rate of random integration any repetitive element was excluded from the homology arms used in the dsDNA donors. Random integration events were excluded by the selection of tagBFP negative cells (FIGS. 1A and 1C and FIGS. 3A and 3D). The efficiency of biallelic knock in for SNCA was 5%. Stepwise enrichment of the double positive population ranged 5%-10% in the first sorting, 95-98 in the second to 99.9-99.9% in the third for all the mutants analyzed.

### Example 6: Enrichment of Bi-Allele Targeted Cells

The positive selection module coupled to fluorescent protein markers allows the genotyping by FACS or fluorescence microscopy for the enrichment or fluorescence guided capture of gene edited lines (such as, homozygous gene corrected edited lines, homozygous mutant genome edited lines, heterozygous mutant genome edited lines and heterozygous gene corrected edited lines but not exclusively).

By the use of double stranded DNA donors (nucleic acid molecules of the invention) with homology arms carrying both (homozygous) or only one (heterozygous) specific sequence and positive selection cassettes containing fluorescent selection modules, the enrichment of gene corrected lines from a population of genome targeted cells is possible based on the fluorescence expression pattern. Examples for editing specific disease patient lines or for creating isogenic control lines for in vitro disease modelling are mentioned below.

### Enrichment of Non-Random Integration Edited Lines

By the use of a fluorescent reporter or a chemical selection located in a different area of the positive selection cassette of the donor, enrichment of cells not carrying this reporter will be avoided since the fluorescent positive cells in this case represent incorrect gene edited lines.

### Enrichment of Homozygous Gene Edited Lines.

By the use of double stranded DNA donors with homology arms carrying both a specific sequence and positive selection cassette containing fluorescent selection modules, the enrichment of gene corrected lines from a population of genome targeted cells is possible based on the fluorescence expression pattern.

### Enrichment of Homozygous Gene Corrected Edited Lines.

By the use of double stranded DNA donors with homology arms carrying wild type alleles and positive selection cassette containing fluorescent selection modules, the enrichment of gene corrected lines from a population of genome targeted cells is possible based on the fluorescence expression pattern.

### Enrichment of Heterozygous Gene Edited Lines.

By the use of double stranded DNA donors with homology arms carrying different specific sequences and positive selection cassette containing fluorescent selection modules, the enrichment of gene corrected lines from a population of genome targeted cells is possible based on the fluorescence expression pattern.

### Enrichment of Heterozygous Mutant Genome Edited Lines.

By the use of double stranded DNA donors with homology arms carrying one of them a mutant allele and the other a wild type allele and both carrying positive selection cassette containing fluorescent selection modules the enrichment of gene heterozygote mutant lines from a population of genome targeted cells is possible based on the fluorescence expression pattern.

### Enrichment of Homozygous Mutant Genome Edited Lines

By the use of double stranded DNA donors with homology arms carrying mutant alleles and positive selection cassette containing fluorescent selection modules the enrichment of gene homozygous mutant lines from a population of genome targeted cells is possible based on the fluorescence expression pattern.

### Items

1. A nucleic acid molecule comprising at least one nucleotide sequence encoding a selection marker indicating homologous recombination when integrated in the sequence of interest comprised in a eukaryotic cell and at least one nucleotide sequence encoding a selection marker indicating heterologous recombination when not integrated in the sequence of interest comprised in said eukaryotic cell, wherein the selection markers when being expressed are optically discriminable, e.g. in FACS or any fluorescence guided capture, and wherein the nucleotide sequence encoding a selection marker indicating homologous recombination in a eukaryotic cell is flanked 5′ and 3′ by nucleotide sequences that are homologous to nucleotide sequences of a nucleic acid sequence of interest comprised by the eukaryotic cell.
2. The nucleic acid molecule of item 1, wherein said nucleic acid sequence of interest comprised by said eukaryotic cell is in the genome of said eukaryotic cell.
3. The nucleic acid molecule of any one of the preceding items, wherein said nucleotide sequence encoding a selection marker indicating homologous recombination and said selection marker indicating heterologous recombination comprises a promoter driving expression of said selection markers.
4. The nucleic acid molecule of item 3, wherein said promoter is constitutive or inducible.
5. The nucleic acid molecule of any one of the preceding items, wherein said nucleotide sequence encoding a selection marker indicating homologous recombination comprises 5′ and 3′ nucleotide sequences which allow excision of said nucleotide sequence encoding said selection marker.
6. The nucleic acid molecule of item 5, wherein said nucleotide sequences which allow excision of said nucleotide sequence encoding said selection marker are selected from loxP sequences, derivatives of loxP sequences, such as Lox511, Lox5171, Lox2272, M2, M3, M7, M11, Lox71 or Lox66 sequences, Cre recombinase binding sites, FRT sequences, derivatives of FRT sequences, such as FRT-G, FRT-H or FRT-F3, FLP recombinase binding sites, terminal repeats, transposase binding sites, derivatives of transposase binding sites, such as terminal repeats, internal terminal repeats, direct repeats, inverted repeats or palindromic repeats, piggybac transposon binding sites, sleeping beauty transposon binding sites, piggybat binding sites, binding site of transposons fused to estrogen receptor, estrogen binding sites or mutational derivatives, binding site of transposons fused to estrogen receptor tamoxifen binding sites, RNA-guided nuclease binding site, Cas9 binding sites, Cpf binding site, nuclease binding sites, TALEN-Fok1 binding sites, zinc finger binding sites or RNA-guided nuclease binding sites.
7. The nucleic acid molecule of any one of the preceding items, wherein said nucleic acid molecule comprises a chemical resistance selection marker selected from neomycin resistance, hygromycin resistance, HPRT1, puromycin resistance, puromycin N-acetyl-transferase, blasticidin resistance, G418 resistance, phleomycin resistance, nourseothricin resistance or chloramphenicol resistance.
8. The nucleic acid molecule of item 7, wherein said chemical resistance selection marker is associated or not associated with said selection marker indicating homologous recombination.
9. The nucleic acid molecule of any one of the preceding items, wherein said nucleotide sequences that are homologous to nucleotide sequences of a nucleic acid sequence of interest comprised by said eukaryotic cell allow homologous recombination with nucleotide sequences of a nucleic acid sequence of interest comprised by said eukaryotic cell.
10. The nucleic acid molecule of item 9, wherein homologous recombination allows depositing a modification into the genome, said modification is selected from a single nucleotide polymorphism, phosphomimetic mutation, phospho null mutation, missense mutation, nonsense mutation, synonymous mutation, insertion, deletion, knock-out or knock-in.
11. The nucleic acid molecule of item 9 or 10, wherein homologous recombination occurs at one allele or at both alleles of said nucleic acid sequence of interest comprised by said eukaryotic cell.
12. The nucleic acid molecule of any one of the preceding items, wherein homologous recombination is induced by TALENs, ZFNs, meganucleases, or CRISPR/Cas.
13. The nucleic acid molecule of item 12, wherein said nucleotide sequences that are homologous to nucleotide sequences of a nucleic acid sequence of interest comprised by said eukaryotic cell do not comprise a target sequence for TALENs, ZFNs, meganucleases, or CRISPR/Cas which mediate homologous recombination.
14. The nucleic acid molecule of any one of the preceding items, wherein said nucleic acid molecule is a vector.
15. The nucleic acid molecule of item 14, wherein said vector is circular or linearized.
16. The nucleic acid molecule of any one of the preceding items, wherein said optical discriminability is different emission wavelength.
17. The nucleic acid molecule of any one of the preceding items, wherein said selection marker indicating homologous recombination and said selection marker indicating heterologous recombination is a fluorescent protein.
18. The nucleic acid molecule of item 17, wherein said fluorescent protein is selected from Sirius, SBFP2, Azurite, EBFP2, mKalama1, mTagBFP2, Aquamarine, ECFP, Cerulean, mCerulean3, SCFP3A, mTurquoise2, CyPet, AmCyan1, mTFP1, MiCy, iLOV, AcGFP1, sfGFP, mEmerald, EGFP, mAzamiGreen, cfSGFP2, ZsGreen, mWasabi, SGFP2, Clover, mClover2, EYFP, mTopaz, mVenus, SYFP2, mCitrine, YPet, ZsYellow1, mPapaya1, mKO, mOrange, mOrange2, mKO2, TurboRFP, mRuby2, eqFP611, DsRed2, mApple, mStrawberry, FusionRed, mRFP1, mCherry, mCherry2, dTOMATO, tdTOMATO, tagBFP, photoactivatable or photoswitchable fluorescent protein.
19. A composition of matter comprising a mixture of at least two different nucleic acid molecules of any one of items 1 to 18, each comprising a different nucleotide sequence encoding a selection marker indicating homologous recombination in said eukaryotic cell.
20. An in vitro method for enriching eukaryotic cells which are modified by homologous recombination, comprising
   (a) subjecting a population of cells transformed with a nucleic acid molecule of any one of items 1 to 18 or a composition of item 19 to means for selecting for said marker indicating heterologous recombination and separate transformed cells expressing said selection marker indicating heterologous recombination in said eukaryotic cell; and
   (b) subjecting the non-separated cells to means for selecting for said marker indicating homologous recombination in order to enrich transformed cells comprising said homologous recombination.
21. The method of item 20, further comprising (c) subjecting said enriched cells to sequencing.
22. The method of item 20 or 21, further comprising removing the nucleotide sequence encoding said marker indicating homologous recombination.
23. An in vitro method for producing eukaryotic cells comprising a modification in its genome introduced by homologous recombination, comprising
   (a) subjecting a population of cells transformed with a nucleic acid molecule of any one of items 1 to 18 or a composition of item 19 to means for selecting for said marker indicating heterologous recombination and separate transformed cells expressing said selection marker indicating heterologous recombination in said eukaryotic cell;
   (b) subjecting the non-separated cells to means for selecting for said marker indicating homologous recombination in order to enrich transformed cells comprising said homologous recombination; and
   (c) sequencing said enriched transformed cells in order to determine whether said enriched transformed cells comprise the desired modification.
24. The method of item 23, further comprising removing the nucleotide sequence encoding said marker indicating homologous recombination.
25. Use of a nucleic acid molecule of any one of items 1 to 18 or a composition of item 19 for enriching eukaryotic cells which are modified by homologous recombination.
26. Use of a nucleic acid molecule of any one of items 1 to 18 or a composition of item 19 for producing eukaryotic cells comprising a modification in its genome introduced by homologous recombination.

1. A composition of matter comprising a mixture of at least two different nucleic acid molecules, each nucleic acid molecule comprising at least one nucleotide sequence encoding a selection marker indicating homologous recombination when integrated in the sequence of interest comprised in a eukaryotic cell and at least one nucleotide sequence encoding a selection marker indicating heterologous recombination when not integrated in the sequence of interest comprised in said eukaryotic cell, wherein the selection markers when being expressed are optically discriminable, e.g. in FACS or any fluorescence guided capture, and wherein the nucleotide sequence encoding a selection marker indicating homologous recombination in a eukaryotic cell is flanked 5′ and 3′ by nucleotide sequences that are homologous to nucleotide sequences of a nucleic acid sequence of interest comprised by the eukaryotic cell, each of the at least two different nucleic acid molecules comprising a different nucleotide sequence encoding a selection marker indicating homologous recombination in said eukaryotic cell.

2. The composition of claim 1, wherein said nucleic acid sequence of interest comprised by said eukaryotic cell is in the genome of said eukaryotic cell.

3. The composition of claim 1, wherein said nucleotide sequence encoding a selection marker indicating homologous recombination and said selection marker indicating heterologous recombination each comprises a promoter driving expression of said selection markers.

4. The composition of claim 3, wherein said promoter is constitutive or inducible.

5. The composition of claim 1, wherein said nucleotide sequence encoding a selection marker indicating homologous recombination comprises 5′ and 3′ nucleotide sequences which allow excision of said nucleotide sequence encoding said selection marker.

6. The composition of claim 5, wherein said nucleotide sequences which allow excision of said nucleotide sequence encoding said selection marker are selected from loxP sequences, derivatives of loxP sequences, such as Lox511, Lox5171, Lox2272, M2, M3, M7, M11, Lox71 or Lox66 sequences, Cre recombinase binding sites, FRT sequences, derivatives of FRT sequences, such as FRT-G, FRT-H or FRT-F3, FLP recombinase binding sites, terminal repeats, transposase binding sites, derivatives of transposase binding sites, such as terminal repeats, internal terminal repeats, direct repeats, inverted repeats or palindromic repeats, piggybac transposon binding sites, sleeping beauty transposon binding sites, piggybat binding sites, binding site of transposons fused to estrogen receptor, estrogen binding sites or mutational derivatives, binding site of transposons fused to estrogen receptor tamoxifen binding sites, RNA-guided nuclease binding site, Cas9 binding sites, Cpf binding site, nuclease binding sites, TALEN-Fok1 binding sites, zinc finger binding sites or RNA-guided nuclease binding sites.

7. The composition of claim 1, wherein said nucleic acid molecule comprises a chemical resistance selection marker selected from neomycin resistance, hygromycin resistance, HPRT1, puromycin resistance, puromycin N-acetyl-transferase, blasticidin resistance, G418 resistance, phleomycin resistance, nourseothricin resistance or chloramphenicol resistance.

8. The composition of claim 7, wherein said chemical resistance selection marker is associated or not associated with said selection marker indicating homologous recombination.

9. The composition of claim 1, wherein said nucleotide sequences that are homologous to nucleotide sequences of a nucleic acid sequence of interest comprised by said eukaryotic cell allow homologous recombination with nucleotide sequences of a nucleic acid sequence of interest comprised by said eukaryotic cell.

10. The composition of claim 9, wherein homologous recombination allows depositing a modification into the genome, said modification is selected from a single nucleotide polymorphism, phosphomimetic mutation, phospho null mutation, missense mutation, nonsense mutation, synonymous mutation, insertion, deletion, knock-out or knock-in.

11. The composition of claim 9, wherein homologous recombination occurs at one allele or at both alleles of said nucleic acid sequence of interest comprised by said eukaryotic cell.

12. The composition of claim 1, wherein homologous recombination is induced by TALENs, ZFNs, meganucleases, or CRISPR/Cas.

13. The composition of matter of claim 12, wherein said nucleotide sequences that are homologous to nucleotide sequences of a nucleic acid sequence of interest comprised by said eukaryotic cell do not comprise a target sequence for TALENs, ZFNs, meganucleases, or CRISPR/Cas which mediate homologous recombination.

14. The composition of claim 1, wherein said nucleic acid molecule is a vector.

15. The composition of claim 14, wherein said vector is circular or linearized.

16. The composition of claim 1, wherein said optical discriminability is different emission wavelength.

17. The composition of claim 1, wherein said selection marker indicating homologous recombination and said selection marker indicating heterologous recombination is a fluorescent protein.

18. The composition of claim 17, wherein said fluorescent protein is selected from Sirius, SBFP2, Azurite, EBFP2, mKalama1, mTagBFP2, Aquamarine, ECFP, Cerulean, mCerulean3, SCFP3A, mTurquoise2, CyPet, AmCyan1, mTFP1, MiCy, iLOV, AcGFP1, sfGFP, mEmerald, EGFP, mAzamiGreen, cfSGFP2, ZsGreen, mWasabi, SGFP2, Clover, mClover2, EYFP, mTopaz, mVenus, SYFP2, mCitrine, YPet, ZsYellow1, mPapaya1, mKO, mOrange, mOrange2, mKO2, TurboRFP, mRuby2, eqFP611, DsRed2, mApple, mStrawberry, FusionRed, mRFP1, mCherry, mCherry2, dTOMATO, tdTOMATO, tagBFP, photoactivatable or photoswitchable fluorescent protein.

19. An in vitro method for enriching eukaryotic cells which are modified by homologous recombination, comprising
(a) subjecting a population of cells transformed with a composition of claim 1 to means for selecting for said marker indicating heterologous recombination and separate transformed cells expressing said selection marker indicating heterologous recombination in said eukaryotic cell; and
(b) subjecting the non-separated cells to means for selecting for said marker indicating homologous recombination in order to enrich transformed cells comprising said homologous recombination; optionally further comprising
(c) subjecting said enriched cells to sequencing; optionally further comprising
(d) removing the nucleotide sequence encoding said marker indicating homologous recombination.

20.-21. (canceled)

22. An in vitro method for producing eukaryotic cells comprising a modification in its genome introduced by homologous recombination, comprising
(a) subjecting a population of cells transformed with a composition of claim 1 to means for selecting for said marker indicating heterologous recombination and separate transformed cells expressing said selection marker indicating heterologous recombination in said eukaryotic cell;
(b) subjecting the non-separated cells to means for selecting for said marker indicating homologous recombination in order to enrich transformed cells comprising said homologous recombination; and
(c) sequencing said enriched transformed cells in order to determine whether said enriched transformed cells comprise the desired modification; optionally further comprising
(d) removing the nucleotide sequence encoding said marker indicating homologous recombination.

23.-25. (canceled)
